(19) United States
(12) Patent Application Publication
(10) Pub. No.: US 2024/0081854 A1
(43) Pub. Date: Mar. 14, 2024

(54) ESTIMATION METHOD AND ESTIMATION DEVICE

(71) Applicant: OLYMPUS MEDICAL SYSTEMS CORP., Tokyo (JP)

(72) Inventor: Yuto HIRABAYASHI, Tokyo (JP)

(21) Appl. No.: 18/460,102

(22) Filed: Sep. 1, 2023

Related U.S. Application Data
(60) Provisional application No. 63/374,489, filed on Sep. 2, 2022.

(51) Int. Cl. A61B 17/32; A61B 1/018; A61B 1/04; A61B 1/06

(52) U.S. Cl. CPC A61B 17/320068; A61B 1/018; A61B 1/043; A61B 1/0638; A61B 2017/00026; A61B 2017/320082

(57) ABSTRACT

Provided is an estimation method executed by a processor of an estimation device. The estimation method includes: acquiring monitor information regarding an electrical characteristic value in an energy treatment tool when non-treatment energy for monitoring is applied to a living tissue from the energy treatment tool; acquiring image information regarding an endoscopic image obtained by imaging the living tissue; and performing estimation regarding thermal invasion in the living tissue based on at least one of the monitor information and the image information and on an output setting value of treatment energy for treating the living tissue with the energy treatment tool.

### CROSS REFERENCES TO RELATED APPLICATIONS

This application is based on and claims priority under 35 U.S.C. § 119 to U.S. Provisional Application No. 63/374,489, filed on Sep. 2, 2022, the entire contents of which are incorporated herein by reference.

### FIELD OF DISCLOSURE

The present disclosure relates to an estimation method and an estimation device.

### BACKGROUND

A treatment system that treats a living tissue by applying treatment energy to the living tissue from an energy treatment tool can be found, for example, in WO 2015/122308 A and WO 2017/187523 A.

In the treatment system described in WO 2015/122308 A, ultrasonic energy is employed as the treatment energy. Then, in the energy treatment system, the treatment (incision) completion of the living tissue is determined by monitoring the behavior of an ultrasonic impedance value.

In addition, in the treatment system described in WO 2017/187523 A, high frequency energy is employed as the treatment energy. Then, in the energy treatment system, the operation of the energy treatment tool is controlled by monitoring the behavior of the impedance value of the living tissue.

### SUMMARY

In some embodiments, provided is an estimation method executed by a processor of an estimation device. The estimation method includes: acquiring monitor information regarding an electrical characteristic value in an energy treatment tool when non-treatment energy for monitoring is applied to a living tissue from the energy treatment tool; acquiring image information regarding an endoscopic image obtained by imaging the living tissue; and performing estimation regarding thermal invasion in the living tissue based on at least one of the monitor information and the image information and on an output setting value of treatment energy for treating the living tissue with the energy treatment tool.

In some embodiments, provided is an estimation method executed by a processor of an estimation device. The estimation method includes: acquiring output information regarding an electrical characteristic value in an energy treatment tool when treatment energy is applied to a living tissue from the energy treatment tool; acquiring image information regarding an endoscopic image obtained by imaging the living tissue; and performing estimation regarding thermal invasion in the living tissue based on the output information and the image information.

In some embodiments, an estimation device includes at least one processor, the processor being configured to: acquire monitor information regarding an electrical characteristic value in an energy treatment tool when non-treatment energy for monitoring is applied to a living tissue from the energy treatment tool; acquire image information regarding an endoscopic image obtained by imaging the living tissue; and perform estimation regarding thermal invasion in the living tissue based on at least one of the monitor information and the image information and on an output setting value of treatment energy for treating the living tissue with the energy treatment tool.

In some embodiments, an estimation device includes at least one processor, the processor being configured to: acquire output information regarding an electrical characteristic value in an energy treatment tool when treatment energy is applied to a living tissue from the energy treatment tool; acquire image information regarding an endoscopic image obtained by imaging the living tissue; and perform estimation regarding thermal invasion in the living tissue based on the output information and the image information.

The above and other features, advantages and technical and industrial significance of this disclosure will be better understood by reading the following detailed description of presently embodiments of the disclosure, when considered in connection with the accompanying drawings.

### BRIEF DESCRIPTION OF THE DRAWINGS

FIG. 1 is a view illustrating a treatment system according to a first embodiment;

FIG. 2 is a view illustrating a transducer unit.

FIG. 3 is a block diagram illustrating a configuration of a control device;

FIG. 4 is a flowchart illustrating a control method executed by a processor;

FIG. 5 is a view for explaining image information (1) to image information (3);

FIG. 6 is a flowchart illustrating a control method according to a second embodiment;

FIG. 7 is a flowchart illustrating a control method according to a third embodiment;

FIG. 8 is a view for explaining image information (6);

FIG. 9 is a view for explaining image information (7);

FIG. 10 is a view illustrating a modification of the third embodiment;

FIG. 11 is a diagram illustrating the modification of the third embodiment; and

FIG. 12 is a flowchart illustrating a control method according to a fourth embodiment.

### DETAILED DESCRIPTION

Hereinafter, modes for carrying out the disclosure (embodiments) will be described with reference to the drawings. Note that the disclosure is not limited by the embodiments described below. Furthermore, in the description of the drawings, the same portions are denoted by the same reference numerals.

### First Embodiment

### Schematic Configuration of Treatment System

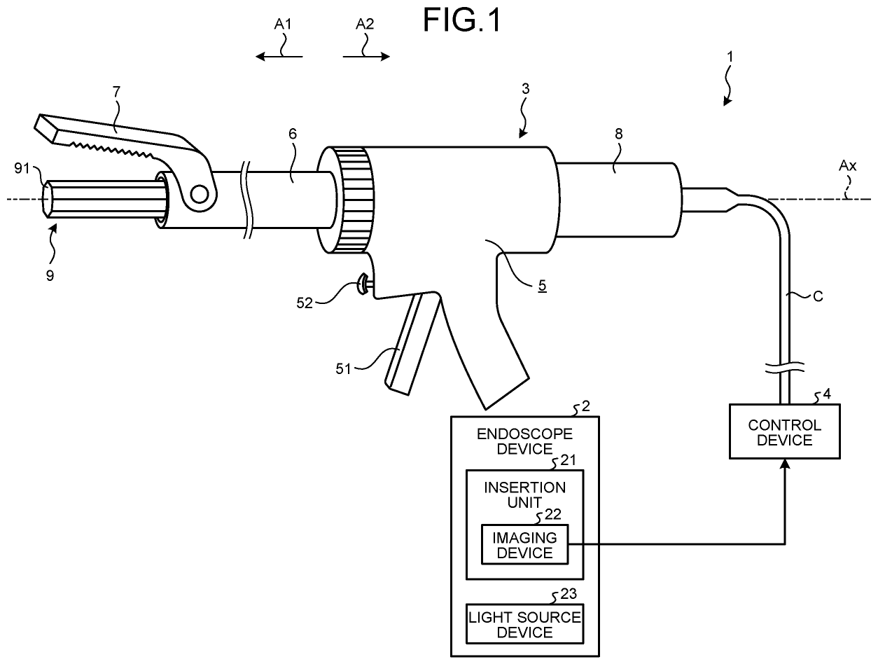

FIG. 1 is a view illustrating a treatment system 1 according to a first embodiment.

The treatment system 1 applies treatment energy to a treatment target region (hereinafter, described as a target region) in a living tissue to treat the target region. In the first embodiment, ultrasonic energy and high frequency energy are employed as the treatment energy. In addition, the treatment is coagulation (sealing) or incision of the target region. Note that as the treatment, the coagulation (sealing) and incision of the target region may be performed simultaneously. As illustrated in FIG. 1, the treatment system 1 includes an endoscope device 2, an energy treatment tool 3, and a control device 4.

### Configuration of Endoscope Device

The endoscope device 2 is partially inserted into a living body, images the inside of the living body, and outputs an image signal (hereinafter, described as an endoscopic image) generated by the imaging. As illustrated in FIG. 1, the endoscope device 2 includes an insertion unit 21, an imaging device 22, and a light source device 23.

The insertion unit 21 is a portion at least a part of which has flexibility and is inserted into the living body.

The light source device 23 supplies illumination light to irradiate the inside of the living body from the distal end of the insertion unit 21. In the first embodiment, the light source device 23 can supply, as the illumination light, at least one light of white light, special light, and excitation light, respectively.

Here, the white light is visible light and is illumination light used in normal light observation.

The special light is illumination light used in special light observation and has a specific wavelength band. In the first embodiment, the illumination light is used in narrow band imaging (NBI). NBI is an observation method in which a capillary vessel and a mucosal surface structure of a mucosal surface layer of a living tissue are enhanced by utilizing the fact that hemoglobin in blood strongly absorbs light in the vicinity of a wavelength of 415 nm. Then, the special light includes first narrow band light having a wavelength band of about 530 nm to 550 nm and second narrow band light having a wavelength band of about 390 nm to 445 nm.

In addition, the excitation light is illumination light used in fluorescence observation. In the first embodiment, the excitation light is excitation light for generating fluorescence from advanced glycation endproducts generated by heat treatment of the energy treatment tool 3 on the target region, and is light having a wavelength band of about 400 nm to 430 nm.

The imaging device 22 is provided at a distal end portion in the insertion unit 21. Then, the imaging device 22 includes an imaging element such as a charge coupled device (CCD) or a complementary metal oxide semiconductor (CMOS) that receives a subject image and converts the subject image into an electric signal, and outputs an endoscopic image generated by imaging the inside of the living body to the control device 4.

Here, in a case where the light source device 23 supplies white light (illumination light), the imaging device 22 generates, as the endoscopic image, a white light image obtained by imaging a living tissue irradiated with the white light.

In addition, in a case where the light source device 23 supplies special light (illumination light), the imaging device 22 generates, as the endoscopic image, a special light image obtained by imaging a living tissue irradiated with the special light.

Furthermore, in a case where the light source device 23 supplies excitation light (illumination light), the imaging device 22 generates, as the endoscopic image, a fluorescence image obtained by imaging fluorescence generated from a living tissue (advanced glycation endproducts) by irradiation of the living tissue with the excitation light.

### Configuration of Energy Treatment Tool

The energy treatment tool 3 is an ultrasonic treatment tool having a bolted Langevin-type transducer (BLT). As illustrated in FIG. 1, the energy treatment tool 3 includes a handle 5, a sheath 6, a jaw 7, a transducer unit 8, and a vibration transmission member 9.

The handle 5 is a portion which an operator holds with their hand. Then, as illustrated in FIG. 1, the handle 5 is provided with an operation knob 51 and an operation button 52.

The sheath 6 has a cylindrical shape. Note that hereinafter, the central axis of the sheath 6 is referred to as a central axis Ax (FIG. 1). In addition, hereinafter, one side along the central axis Ax is referred to as a distal end side A1 (FIG. 1), and the other side is referred to as a proximal end side A2 (FIG. 1). Then, the sheath 6 is attached to the handle 5 in a state where a part of the proximal end side A2 is inserted into the handle 5 from the distal end side A1 of the handle 5.

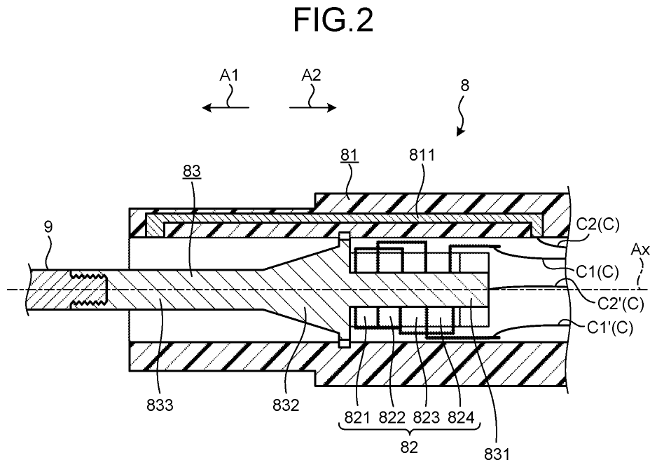

FIG. 2 is a view illustrating the transducer unit 8. Specifically, FIG. 2 is a cross-sectional view of the transducer unit 8 taken along a plane including the central axis Ax.

As illustrated in FIG. 2, the transducer unit 8 includes a transducer case 81, an ultrasound transducer 82, and a horn 83.

The transducer case 81 extends linearly along the central axis Ax, and is attached to the handle 5 in a state where a part of the distal end side A1 is inserted into the handle 5 from the proximal end side A2 of the handle 5.

The ultrasound transducer 82 is housed inside the transducer case 81 and generates ultrasonic vibration under the control of the control device 4. In the first embodiment, the ultrasonic vibration is a BLT including a plurality of piezoelectric elements 821 to 824 stacked along the central axis Ax. In the first embodiment, the piezoelectric elements are configured by four piezoelectric elements 821 to 824, but the number of piezoelectric elements is not limited to four, and another number of piezoelectric elements may be used.

The horn 83 is housed inside the transducer case 81 and expands the amplitude of the ultrasonic vibration generated by the ultrasound transducer 82. The horn 83 has an elongated shape extending linearly along the central axis Ax. As illustrated in FIG. 2, the horn 83 has a configuration in which a first attachment portion 831, a cross-sectional area change portion 832, and a second attachment portion 833 are arranged from the proximal end side A2 to the distal end side A1.

The first attachment portion 831 is a portion to which the ultrasound transducer 82 is attached.

The cross-sectional area change portion 832 has a shape in which the cross-sectional area decreases toward the distal end side A1, and is a portion that enlarges the amplitude of the ultrasonic vibration.

The second attachment portion 833 is a portion to which the end portion of the vibration transmission member 9 on the proximal end side A2 is attached.

The jaw 7 and the vibration transmission member 9 grip the target region and apply ultrasonic energy and high frequency energy to the target region to treat the target region.

Specifically, the jaw 7 is made of a conductive material such as metal, and is rotatably attached to the end portion of the sheath 6 on the distal end side A1. Then, the jaw 7 grips the target region with a treatment unit 91 (FIG. 1) configuring the vibration transmission member 9.

Although not specifically illustrated, an opening and closing mechanism for opening and closing the jaw 7 with respect to the treatment unit 91 according to the operation of the operation knob 51 by the operator is provided inside the handle 5 and the sheath 6 described above. In addition, in the jaw 7, a pad (not illustrated) made of resin is attached to a surface facing the treatment unit 91. Since the pad has an electrical insulation property, the pad has a function of preventing a short circuit between the jaw 7 and the vibration transmission member 9. In addition, the pad has a function of preventing that the vibration transmission member 9 which is ultrasonically vibrating is damaged by colliding with the jaw 7 when the incision of the target region by the ultrasonic vibration is completed.

The vibration transmission member 9 is made of a conductive material such as metal, and has an elongated shape extending linearly along the central axis Ax. As illustrated in FIG. 1, the vibration transmission member 9 is inserted into the sheath 6 in a state where the treatment unit 91 which is an end portion on the distal end side A1 protrudes to the outside. In addition, as illustrated in FIG. 2, the end portion of the vibration transmission member 9 on the proximal end side A2 is connected to the second attachment portion 833. Then, the vibration transmission member 9 transmits the ultrasonic vibration, which has been generated by the ultrasound transducer 82 and passed through the horn 83, from the proximal end side A2 to the end portion on the distal end side A1, and applies the ultrasonic vibration to the target region gripped between the treatment unit 91 and the jaw 7 to treat the target region. That is, the target region is treated by application of ultrasonic energy from the end portion on the distal end side A1.

### Configuration of Control Device

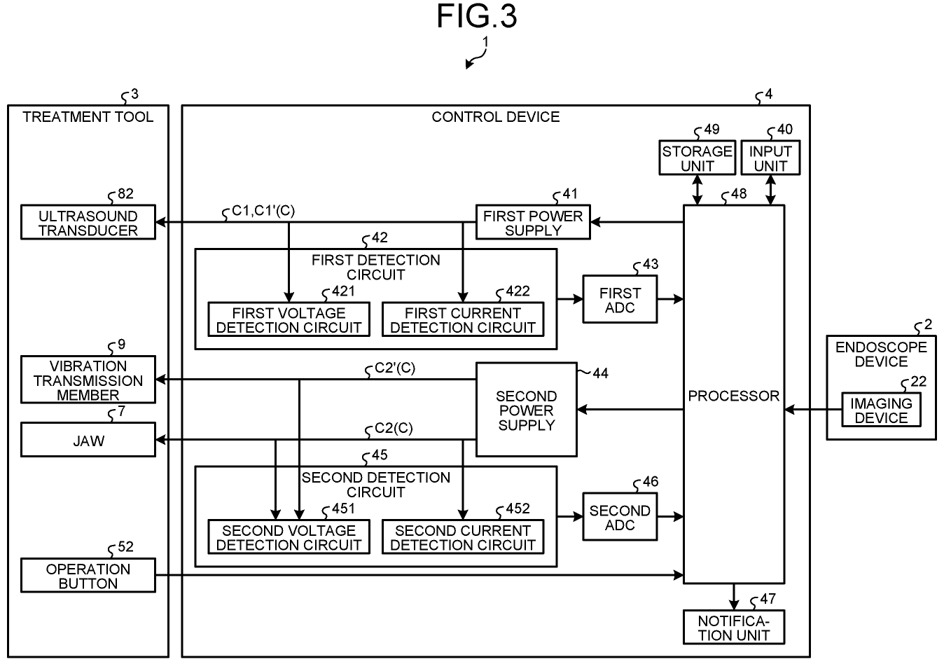

FIG. 3 is a block diagram illustrating a configuration of the control device 4.

The control device 4 corresponds to an estimation device. The control device 4 is electrically connected to the energy treatment tool 3 by an electric cable C (FIG. 1) and comprehensively controls the operation of the energy treatment tool 3. As illustrated in FIG. 3, the control device 4 includes a first power supply 41, a first detection circuit 42, a first analog-to-digital converter (ADC) 43, a second power supply 44, a second detection circuit 45, a second ADC 46, a notification unit 47, a processor 48, a storage unit 49, and an input unit 40.

Here, as illustrated in FIG. 2, a pair of transducer lead wires C1 and C1′ configuring the electric cable C is joined to the ultrasound transducer 82. Note that in FIG. 3, only one pair of transducer lead wires C1 and C1′ is illustrated for convenience of description.

Then, the first power supply 41 outputs a first drive signal, which is power for generating ultrasonic vibration, to the ultrasound transducer 82 via the pair of transducer lead wires C1 and C1′ under the control of the processor 48. As a result, the ultrasound transducer 82 generates ultrasonic vibration.

Hereinafter, for convenience of description, the first drive signal output from the first power supply 41 to the ultrasound transducer 82 is referred to as a first input drive signal, and a signal obtained by changing the first input drive signal according to the frequency response of the energy treatment tool 3 (ultrasound transducer 82) is referred to as a first output drive signal.

The first detection circuit 42 includes a first voltage detection circuit 421 which is a voltage sensor which detects a voltage value and a first current detection circuit 422 which is a current sensor which detects a current value, and detects a US signal (analog signal) corresponding to the first output drive signal over time. The US signal corresponds to “an electrical characteristic value in an energy treatment tool”.

Specifically, examples of the US signal include a current value (hereinafter, described as US current) in the first output drive signal, a voltage value (hereinafter, described as US voltage) in the first output drive signal, a power value (hereinafter, described as US power) in the first output drive signal, an impedance value (hereinafter, described as a US impedance value) calculated from the US current and the US voltage, and a frequency (hereinafter, described as US frequency) of the US current or the US voltage.

The first ADC 43 converts the US signal (analog signal) output from the first detection circuit 42 into a digital signal. Then, the first ADC 43 outputs the converted US signal (digital signal) to the processor 48.

Here, as illustrated in FIG. 2, the transducer case 81 is provided with a first conductive portion 811 extending from the end portion on the proximal end side A2 to the end portion on the distal end side A1. In addition, although not specifically illustrated, the sheath 6 is provided with a second conductive portion extending from the end portion on the proximal end side A2 to the end portion on the distal end side A1 and electrically connects the first conductive portion 811 and the jaw 7. Further, a high frequency lead wire C2 configuring the electric cable C is joined to the end portion of the first conductive portion 811 on the proximal end side A2. In addition, a high frequency lead wire C2′ configuring the electric cable C is joined to the first attachment portion 831.

Under the control of the processor 48, the second power supply 44 outputs a second drive signal, which is high frequency power, to the jaw 7 and the vibration transmission member 9 via the pair of high frequency lead wires C2 and C2′, the first conductive portion 811, the second conductive portion, and the horn 83. As a result, a high frequency current flows through the target region gripped between the jaw 7 and the treatment unit 91. That is, high frequency energy is applied to the target region. Then, in the target region, Joule heat is generated by the high frequency current flowing therethrough, and the target region is treated.

As described above, each of the jaw 7 and the treatment unit 91 functions as an electrode.

Hereinafter, for convenience of description, the second drive signal output from the second power supply 44 to the jaw 7 and the vibration transmission member 9 is referred to as a second input drive signal, and a signal obtained by changing the second input drive signal according to the frequency response of the energy treatment tool 3 is referred to as a second output drive signal.

The second detection circuit 45 includes a second voltage detection circuit 451 which is a voltage sensor which detects a voltage value and a second current detection circuit 452 which is a current sensor which detects a current value, and detects an HF signal (analog signal) corresponding to the second output drive signal over time. The HF signal corresponds to “an electrical characteristic value in an energy treatment tool”.

Specifically, examples of the HF signal include a current value (hereinafter, described as a HF current) in the second output drive signal, a voltage value (hereinafter, described as a HF voltage) in the second output drive signal, a power value (hereinafter, described as HF power) in the second output drive signal, an impedance value (hereinafter, described as a HF impedance value) calculated from the HF current and the HF voltage, and a phase difference (hereinafter, described as a HF phase difference) between the HF current and the HF voltage.

The second ADC 46 converts the HF signal (analog signal) output from the second detection circuit 45 into a digital signal. Then, the second ADC 46 outputs the converted HF signal (digital signal) to the processor 48.

The notification unit 47 notifies predetermined information under the control of the processor 48. Examples of the notification unit 47 include a light emitting diode (LED) which notifies predetermined information by lighting, blinking, or a color at the time of lighting, a display device which displays predetermined information, and a speaker which outputs predetermined information by voice. Note that the notification unit 47 may be provided in the control device 4 as illustrated in FIG. 3, or may be provided in the energy treatment tool 3.

The processor 48 includes a controller such as a central processing unit (CPU) and a micro processing unit (MPU), or an integrated circuit such as an application specific integrated circuit (ASIC) and a field programmable gate array (FPGA), and controls the entire operation of the treatment system 1.

Note that detailed functions of the processor 48 will be described in “Control Method Executed by Processor” described later.

The storage unit 49 stores various programs executed by the processor 48, information for processing of the processor 48, and the like. Examples of the information for the processing of the processor 48 include setting values of the first and second input drive signals, a learning model for object identification, and a learning model for estimation. The setting values of the first and second input drive signals correspond to “an output setting value of treatment energy”.

Note that details of the learning model for object identification and the learning model for estimation will be described in “Control Method Executed by Processor” described later.

The input unit 40 includes a keyboard, a mouse, a switch, a touch panel, and the like, and receives a user operation by the operator or the like. Examples of the user operation include an input operation of the setting values of the first and second input drive signals described above. Then, the input unit 40 outputs, to the processor 48, an operation signal corresponding to the user operation.

### Control Method Executed by Processor

Next, a control method executed by the processor 48 will be described. The control method corresponds to an estimation method.

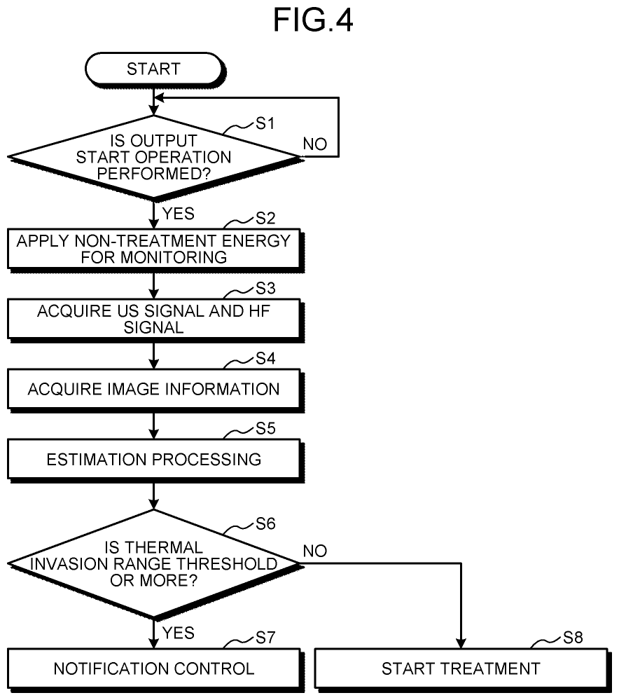

FIG. 4 is a flowchart illustrating the control method executed by the processor 48.

First, the processor 48 constantly monitors whether or not the operator has pressed the operation button 52 (output start operation) (step S1).

When it is determined that there is the output start operation (step S1: Yes), the processor 48 controls the operations of the first and second power supplies 41 and 44. Then, the first power supply 41 outputs the first input drive signal for monitoring to the ultrasound transducer 82 for a certain period of time. Similarly, the second power supply 44 outputs the second input drive signal to the jaw 7 and the vibration transmission member 9 for a certain period of time. As a result, non-treatment energy (ultrasonic energy and high frequency energy) for monitoring is applied to the target region gripped between the jaw 7 and the treatment unit 91 (step S2).

Here, the first and second input drive signals for monitoring are first and second drive signals for applying non-treatment energy for monitoring, which is treatment energy (ultrasonic energy and high frequency energy) to such an extent that a target region is not thermally denatured, to the target region.

While the non-treatment energy for monitoring is applied to the target region in step S2, the processor 48 controls the operations of the first and second detection circuits 42 and 45, and acquires the US signal and the HF signal detected by the first and second detection circuits 42 and 45 (step S3).

After step S3, the processor 48 acquires image information regarding the endoscopic image generated by imaging the target region by the imaging device 22 (step S4).

Here, examples of the image information include the following image information (1) to image information (3).

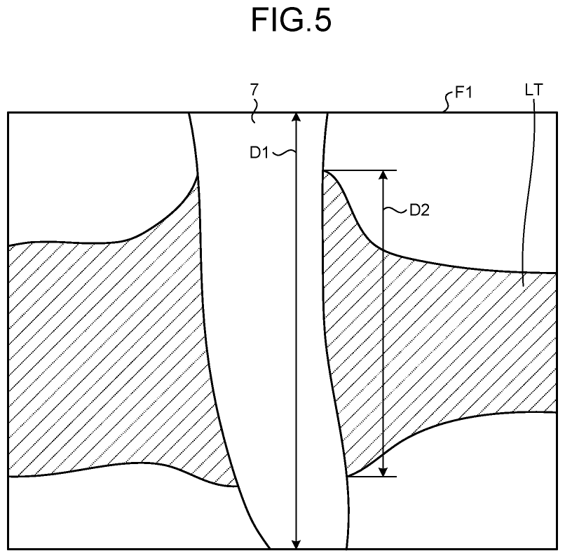

FIG. 5 is a view for explaining the image information (1) to the image information (3). Specifically, in FIG. 5, reference numeral “F1” denotes the endoscopic image generated by the imaging device 22. In addition, a reference sign “LT” indicating a hatched portion is the target region.

The image information (1) is information calculated by the processor 48 on the basis of the endoscopic image F1 as follows.

Specifically, the processor 48 recognizes at least one of the jaw 7 and the treatment unit 91 included as subjects in the endoscopic image F1 and a target region LT gripped between the jaw 7 and the treatment unit 91 by image recognition using the learning model for object identification stored in the storage unit 49. Then, the processor 48 calculates, as the image information (1), a ratio of the length D2 (FIG. 5) of the target region LT gripped between the jaw 7 and the treatment unit 91 to the entire length D1 (FIG. 5) of at least one of the jaw 7 and the treatment unit 91.

In a case where the above-described ratio is large, when treatment energy is applied to the target region LT from the jaw 7 and the treatment unit 91, heat is easily diffused in the target region LT. On the other hand, in a case where the ratio is small, when treatment energy is applied to the target region LT from the jaw 7 and the treatment unit 91, heat is intensively applied to the target region LT. That is, the image information (1) is information that enables estimation of how heat is transferred to the target region LT. In other words, the image information (1) is information that enables estimation of thermal invasion at the target region LT.

The learning model for object identification is a learning model generated by performing machine learning using teacher data in which the endoscopic image obtained by imaging the jaw 7, the treatment unit 91, and the target region LT is associated with the positions of the jaw 7, the treatment unit 91, and the target region LT included as subjects in the endoscopic image.

Here, the learning model for object identification includes a neural network in which each layer has one or a plurality of nodes. In addition, the type of machine learning is not particularly limited, but for example, it is adequate if teacher data is prepared in which a plurality of endoscopic images are associated with the positions of the jaw 7, the treatment unit 91, and the target region LT included as subjects in the plurality of endoscopic images, and the teacher data is input to a calculation model based on a multilayer neural network for learning. Furthermore, as a method of the machine learning, for example, a method based on a deep neural network (DNN) of a multilayer neural network such as a convolutional neural network (CNN) or a 3D-CNN is used. Furthermore, as a method of the machine learning, a method based on a recurrent neural network (RNN), a long short-term memory unit (LSTM) obtained by extending the RNN, or the like may be used.

The image information (2) is information calculated by the processor 48 on the basis of the endoscopic image F1 as follows.

Specifically, the processor 48 recognizes the target region LT gripped between the jaw 7 and the treatment unit 91 included as subjects in the endoscopic image F1 by image recognition using the learning model for object identification stored in the storage unit 49. Then, the processor 48 sets, as the image information (2), the color information (pixel value (RGB value)) of the pixel corresponding to the target region LT in the endoscopic image F1.

The color information varies depending on the tissue type (for example, liver, blood vessel, intestinal tract, and the like) of the target region LT. In addition, when treatment energy is applied to the target region LT from the jaw 7 and the treatment unit 91, how heat is transferred to the target region LT varies depending on the tissue type of the target region LT. That is, the image information (2) is information that enables estimation of how heat is transferred to the target region LT. In other words, the image information (2) is information that enables estimation of thermal invasion at the target region LT.

The image information (3) is information calculated by the processor 48 on the basis of the endoscopic image F1 as follows.

Specifically, the processor 48 recognizes the target region LT gripped between the jaw 7 and the treatment unit 91 included as subjects in the endoscopic image F1 by image recognition using the learning model for object identification stored in the storage unit 49. Then, the processor 48 calculates, as the image information (3), tissue structure information indicating the tissue structure of the target region LT on the basis of the region corresponding to the target region LT in the endoscopic image F1.

Here, examples of the tissue structure information include frequency feature data (information corresponding to surface roughness, pattern, and the like) obtained by edge extraction or Fourier transform for the above-described region, and internal running information of blood vessels and blood flows in a deep portion of a mucosa when the endoscopic image F1 is a special light image. Similarly to the image information (2), the tissue structure information varies depending on the tissue type of the target region LT. That is, the image information (3) is information that enables estimation of how heat is transferred to the target region LT. In other words, the image information (3) is information that enables estimation of thermal invasion at the target region LT.

After step S4, the processor 48 executes estimation processing (step S5).

Specifically, in step S5, by using the learning model for estimation stored in the storage unit 49, the processor 48 uses, as input data, at least one of thirteen pieces of information of monitor information (1) to monitor information (10) described below and the image information (1) to the image information (3) and the setting values of the first and second input drive signals stored in the storage unit 49, and outputs (estimates), as output data, the thermal invasion range of the target region LT. The thermal invasion range indicates how far the thermal invasion occurs from the jaw 7 and the treatment unit 91 in the target region LT.

The monitor information (1) is the US current among the US signals acquired in step S3 when the non-treatment energy for monitoring is applied to the target region LT.

The monitor information (2) is the US voltage among the US signals acquired in step S3 when the non-treatment energy for monitoring is applied to the target region LT.

The monitor information (3) is the US power among the US signals acquired in step S3 when the non-treatment energy for monitoring is applied to the target region LT.

The monitor information (4) is the US impedance value among the US signals acquired in step S3 when the non-treatment energy for monitoring is applied to the target region LT.

The monitor information (5) is the US frequency among the US signals acquired in step S3 when the non-treatment energy for monitoring is applied to the target region LT.

The monitor information (6) is the HF current among the HF signals acquired in step S3 when the non-treatment energy for monitoring is applied to the target region LT.

The monitor information (7) is the HF voltage among the HF signals acquired in step S3 when the non-treatment energy for monitoring is applied to the target region LT.

The monitor information (8) is the HF power among the HF signals acquired in step S3 when the non-treatment energy for monitoring is applied to the target region LT.

The monitor information (9) is the HF impedance value among the HF signals acquired in step S3 when the non-treatment energy for monitoring is applied to the target region LT.

The monitor information (10) is the HF phase difference among the HF signals acquired in step S3 when the non-treatment energy for monitoring is applied to the target region LT.

The monitor information (1) to the monitor information (10) varies depending on the tissue type of the target region LT similarly to the image information (2). That is, the monitor information (1) to the monitor information (10) are information that enables estimation of how heat is transferred to the target region LT. In other words, the monitor information (1) to the monitor information (10) are information that enables estimation of thermal invasion at the target region LT.

The learning model for estimation is a learning model generated by machine learning using teacher data in which at least one of thirteen pieces of information of the monitor information (1) to the monitor information (10), which are acquired when the non-treatment energy for monitoring is applied to the target region LT, and the image information (1) to the image information (3), the setting values of the first and second input drive signals, and the thermal invasion range in the target region LT in a case where the treatment energy according to the setting values of the first and second input drive signals is applied to the target region LT are associated with each other.

Here, the learning model for estimation includes a neural network in which each layer has one or a plurality of nodes. In addition, the type of machine learning is not particularly limited, but for example, it is sufficient if teacher data is prepared in which at least one of thirteen pieces of information of the monitor information (1) to the monitor information (10), which are acquired when the non-treatment energy for monitoring is applied to the target region LT, and the image information (1) to the image information (3), the setting values of the first and second input drive signals, and the thermal invasion range in the target region LT in a case where the treatment energy according to the setting values of the first and second input drive signals is applied to the target region LT are associated with each other, and the learning is performed by inputting the teacher data to the calculation model based on the multilayer neural network. Furthermore, as a method of the machine learning, for example, a method based on DNN of a multilayer neural network such as CNN or 3D-CNN is used. Furthermore, as a method of the machine learning, a method based on a recurrent neural network (RNN), an LSTM obtained by extending an RNN, or the like may be used.

After step S5, the processor 48 determines whether or not the thermal invasion range estimated in step S5 is equal to or more than a threshold (step S6).

In a case where it is determined that the thermal invasion range is equal to or more than the threshold (step S6: Yes), the processor 48 controls the operation of the notification unit 47 without starting the treatment of the target region LT, and causes the notification unit 47 to notify that the thermal invasion range is equal to or more than the threshold (step S7).

On the other hand, in a case where it is determined that the thermal invasion range is less than the threshold (step S6: No), the processor 48 starts the treatment of the target region LT (step S8). Specifically, the processor 48 controls the operations of the first and second power supplies 41 and 44, and causes the first and second power supplies 41 and 44 to output the first and second input drive signals of the setting values stored in the storage unit 49. As a result, the treatment energy (ultrasonic energy and high frequency energy) corresponding to the setting values of the first and second input drive signals is applied to the target region LT gripped between the jaw 7 and the treatment unit 91.

According to the first embodiment described above, the following effects are obtained.

Since the control method (estimation method) executed by the processor 48 according to the first embodiment estimates the thermal invasion range as described above, it is possible to appropriately estimate how far the thermal invasion occurs from the jaw 7 and the treatment unit 91 in the target region LT. That is, the target region LT can be appropriately treated by the estimation.

In particular, since the thermal invasion range can be estimated before the treatment energy is applied to the target region LT, the target region LT can be appropriately treated without performing the treatment in which the thermal invasion is predicted to occur unnecessarily.

### Second Embodiment

Next, a second embodiment will be described.

In the following description, the same reference numerals are given to the same configurations as those of the first embodiment described above, and a detailed description thereof will be omitted or simplified.

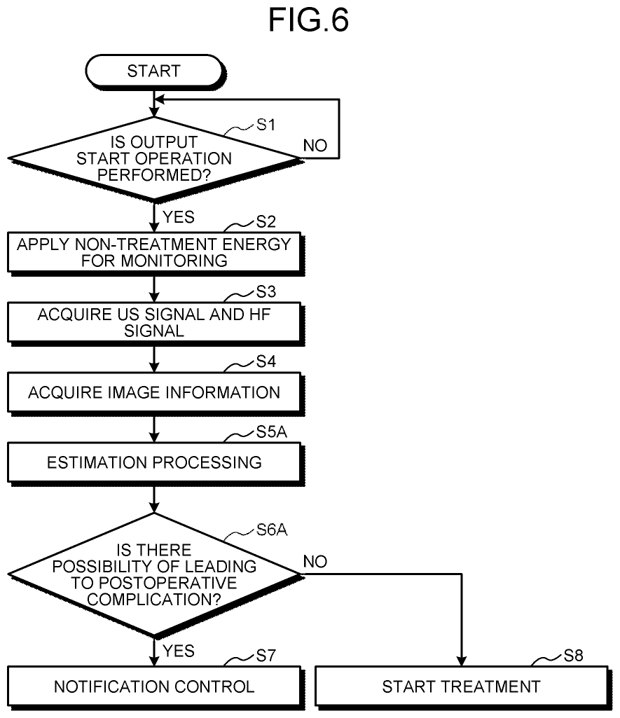

FIG. 6 is a flowchart illustrating a control method according to the second embodiment.

The second embodiment is different from the first embodiment in the estimation processing executed by the processor 48. That is, in the control method according to the second embodiment, as illustrated in FIG. 6, steps S5A and S6A are adopted instead of steps S5 and S6 in the control method described in the first embodiment described above. Hereinafter, only steps S5A and S6A will be mainly described.

Step S5A is executed after step S4.

Specifically, in step S5A, by using the learning model for estimation stored in the storage unit 49, the processor 48 uses, as input data, at least one of thirteen pieces of information of the monitor information (1) to the monitor information (10) and the image information (1) to the image information (3), and the setting values of the first and second input drive signals stored in the storage unit 49, and outputs (estimates), as output data, a possibility of leading to a postoperative complication.

Here, the learning model for estimation according to the second embodiment is different from the learning model for estimation described in the first embodiment described above.

The learning model for estimation according to the second embodiment is a learning model generated by machine learning using teacher data in which at least one of thirteen pieces of information of the monitor information (1) to the monitor information (10), which are acquired when the non-treatment energy for monitoring is applied to the target region LT, and the image information (1) to the image information (3), the setting values of the first and second input drive signals, and the possibility of leading to a postoperative complication in a case where the treatment energy according to the setting values of the first and second input drive signals is applied to the target region LT are associated with each other.

Here, the learning model for estimation according to the second embodiment includes a neural network in which each layer includes one or a plurality of nodes. In addition, the type of machine learning is not particularly limited, but for example, it is sufficient if teacher data is prepared in which at least one of thirteen pieces of information of the monitor information (1) to the monitor information (10), which are acquired when the non-treatment energy for monitoring is applied to the target region LT, and the image information (1) to the image information (3), the setting values of the first and second input drive signals, and the possibility of leading to a postoperative complication in a case where the treatment energy according to the setting values of the first and second input drive signals is applied to the target region LT are associated with each other, and the learning is performed by inputting the teacher data to the calculation model based on the multilayer neural network. Furthermore, as a method of the machine learning, for example, a method based on DNN of a multilayer neural network such as CNN or 3D-CNN is used. Furthermore, as a method of the machine learning, a method based on a recurrent neural network (RNN), an LSTM obtained by extending an RNN, or the like may be used.

Then, in a case where in step S5A, the processor 48 estimates that there is the possibility of leading to a postoperative complication (step S6A: Yes), the process proceeds to step S7.

On the other hand, in a case where in step S5A, the processor 48 estimates that there is no possibility of leading to a postoperative complication (step S6A: No), the process proceeds to step S8.

According to the second embodiment described above, the following effects are obtained.

Since the control method (estimation method) executed by the processor 48 according to the second embodiment estimates the possibility of leading to a postoperative complication as described above, the possibility of leading to a postoperative complication can be appropriately estimated.

In particular, since the possibility of leading to a postoperative complication can be estimated before the treatment energy is applied to the target region LT, the target region LT can be appropriately treated without performing the treatment predicted to lead to a postoperative complication.

### Third embodiment

Next, a third embodiment will be described.

In the following description, the same reference numerals are given to the same configurations as those of the first embodiment described above, and a detailed description thereof will be omitted or simplified.

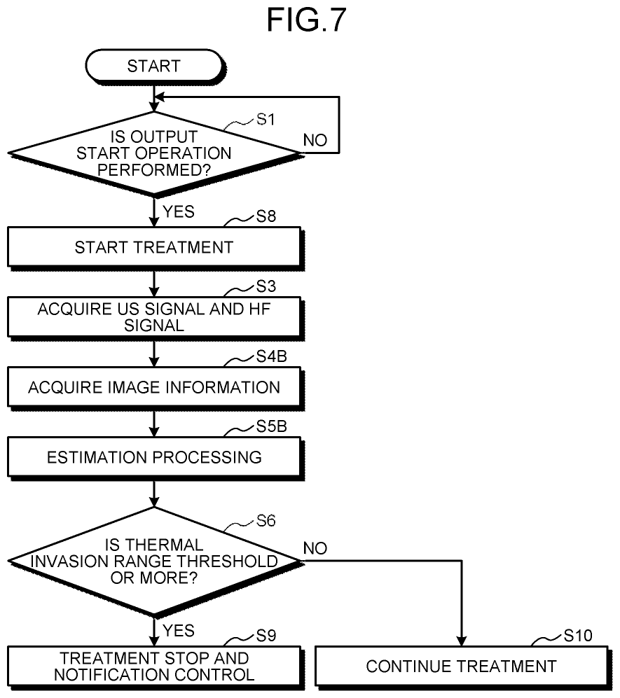

FIG. 7 is a flowchart illustrating a control method according to the third embodiment.

As illustrated in FIG. 7, the third embodiment is different from the first embodiment in the control method executed by the processor 48.

Specifically, in the control method according to the third embodiment, the processing order of step S8 is changed, steps S4B and S5B are adopted instead of steps S4 and S5, and steps S9 and S10 are added. Hereinafter, only steps S8, S4B, S5B, S9, and S10 will be mainly described. Step S8 is executed in a case where it is determined that the output start operation has been performed (step S1: Yes). Thereafter, the processor 48 proceeds to step S3.

Specifically, in step S8, the processor 48 controls the operations of the first and second power supplies 41 and 44, and causes the first and second power supplies 41 and 44 to output the first and second input drive signals of the setting values stored in the storage unit 49. As a result, the treatment energy (ultrasonic energy and high frequency energy) corresponding to the setting values of the first and second input drive signals is applied to the target region LT gripped between the jaw 7 and the treatment unit 91.

Step S4B is executed after step S3.

Specifically, in step S4B, the processor 48 acquires image information regarding the endoscopic image F1 generated by imaging the target region LT by the imaging device 22.

Here, examples of the image information include the following image information (4) to image information (7).

The image information (4) is information calculated by the processor 48 on the basis of the endoscopic image F1 as follows.

Specifically, the processor 48 recognizes at least one of the jaw 7 and the treatment unit 91 included as subjects in the endoscopic image F1 and a target region LT gripped between the jaw 7 and the treatment unit 91 by image recognition using the learning model for object identification stored in the storage unit 49. Then, the processor 48 calculates, as the image information (4), a difference (tissue contraction amount) between the length D2 (FIG. 5) of the target region LT before the treatment energy is applied to the target region LT in step S8 and the length D2 after a lapse of a specific time from the start of the application of the treatment energy in step S8.

The tissue contraction amount varies depending on the tissue type of the target region LT, the amount of heat input to the target region LT, and the like. That is, the image information (4) is information that enables estimation of thermal invasion at the target region LT.

The image information (5) is information calculated by the processor 48 on the basis of the endoscopic image F1 as follows.

Specifically, the processor 48 recognizes at least one of the jaw 7 and the treatment unit 91 included as subjects in the endoscopic image F1 and a target region LT gripped between the jaw 7 and the treatment unit 91 by image recognition using the learning model for object identification stored in the storage unit 49. Then, the processor 48 calculates, as the image information (5), the time (contraction start time) from the start of the application of the treatment energy to the target region LT in step S8 to the change in the length D2 of the target region LT before the start of the application of the treatment energy.

The contraction start time varies depending on the tissue type of the target region LT, the amount of heat input to the target region LT, and the like. That is, the image information (5) is information that enables estimation of thermal invasion at the target region LT.

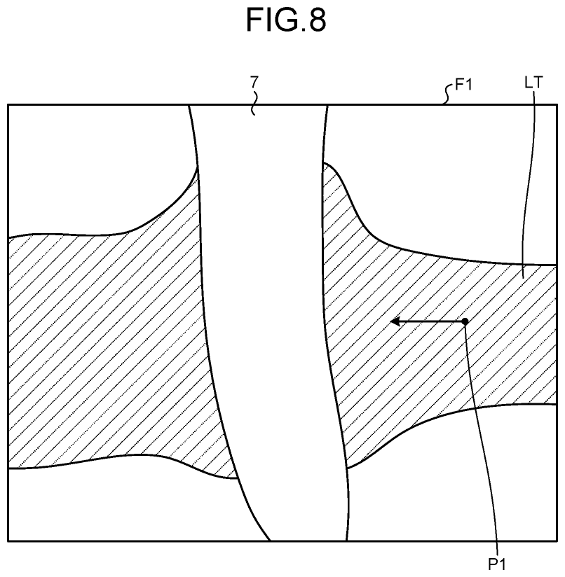

FIG. 8 is a view corresponding to FIG. 5, and is a view for explaining the image information (6).

The image information (6) is information calculated by the processor 48 on the basis of the endoscopic image F1 as follows.

Specifically, the processor 48 recognizes at least one of the jaw 7 and the treatment unit 91 included as subjects in the endoscopic image F1 and a target region LT gripped between the jaw 7 and the treatment unit 91 by image recognition using the learning model for object identification stored in the storage unit 49. Then, the processor 48 focuses on the specific pixel position P1 (FIG. 8) in the region corresponding to the target region LT in the endoscopic image F1, and calculates, as the image information (6), the movement amount by which the pixel position P1 moves toward at least one of the jaw 7 and the treatment unit 91 until a specific time elapses after the application of the treatment energy to the target region LT is started in step S8.

The movement amount has the same characteristics as the above-described tissue contraction amount.

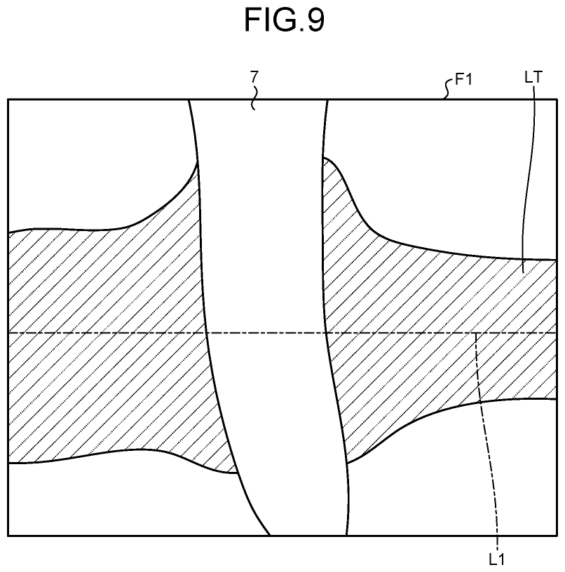

FIG. 9 is a view corresponding to FIG. 5, and is a view for explaining the image information (7).

The image information (7) is information calculated by the processor 48 on the basis of the endoscopic image F1 as follows.

Specifically, the processor 48 recognizes at least one of the jaw 7 and the treatment unit 91 included as subjects in the endoscopic image F1 and a target region LT gripped between the jaw 7 and the treatment unit 91 by image recognition using the learning model for object identification stored in the storage unit 49. In addition, the processor 48 focuses on the variation in color information (pixel values (RGB values)) of pixels on a line L1 (FIG. 9) orthogonal to the longitudinal direction (in FIG. 9, a vertical direction) of the region corresponding to at least one of the jaw 7 and the treatment unit 91 among the pixels corresponding to the target region LT in the endoscopic image F1. Then, the processor 48 calculates, as the image information (7), a change amount of the variation until a specific time elapses since the application of the treatment energy to the target region LT is started in step S8.

When tension is generated by the contraction force of the target region LT, the unevenness of the surface of the target region LT is reduced, so that the change amount of the variation decreases. That is, the change amount of the variation has the same characteristics as the above-described tissue contraction amount.

After step S4B, by using the learning model for estimation stored in the storage unit 49, the processor 48 uses, as input data, at least one of fifteen pieces of information of output information (1) to output information (11) described below and the image information (4) to the image information (7) and outputs (estimates), as output data, the thermal invasion range in the target region LT (step S5B). The thermal invasion range indicates how far the thermal invasion occurs from the jaw 7 and the treatment unit 91 in the target region LT. Thereafter, the processor 48 proceeds to step S6.

The output information (1) is the elapsed time from the start of application of the treatment energy to the target region LT in step S8.

The output information (2) is the US current among the US signals acquired in step S3 when the treatment energy is applied to the target region LT.

The output information (3) is the US voltage among the US signals acquired in step S3 when the treatment energy is applied to the target region LT.

The output information (4) is the US power among the US signals acquired in step S3 when the treatment energy is applied to the target region LT.

The output information (5) is the US impedance value among the US signals acquired in step S3 when the treatment energy is applied to the target region LT.

The output information (6) is the US frequency among the US signals acquired in step S3 when the treatment energy is applied to the target region LT.

The output information (7) is the HF current among the HF signals acquired in step S3 when the treatment energy is applied to the target region LT.

The output information (8) is the HF voltage among the HF signals acquired in step S3 when the treatment energy is applied to the target region LT.

The output information (9) is the HF power among the HF signals acquired in step S3 when the treatment energy is applied to the target region LT.

The output information (10) is the HF impedance value among the HF signals acquired in step S3 when the treatment energy is applied to the target region LT.

The output information (11) is the HF phase difference among the HF signals acquired in step S3 when the treatment energy is applied to the target region LT.

The output information (1) to the output information (11) are information that enables estimation of the amount of heat input to the target region LT. That is, the output information (1) to the output information (11) are information that enables estimation of thermal invasion at the target region LT.

Here, the learning model for estimation according to the third embodiment is different from the learning model for estimation described in the first embodiment described above.

The learning model for estimation according to the third embodiment is a learning model generated by machine learning using teacher data in which at least one of fifteen pieces of information of the output information (1) to the output information (11), which are acquired when the treatment energy is applied to the target region LT, and the image information (4) to the image information (7) is associated with the thermal invasion range in the target region LT when the treatment energy is applied to the target region LT.

Here, the learning model for estimation according to the third embodiment includes a neural network in which each layer includes one or a plurality of nodes. In addition, the type of machine learning is not particularly limited, but for example, it is sufficient if teacher data is prepared in which at least one of fifteen pieces of information of the output information (1) to the output information (11), which are acquired when treatment energy is applied to the target region LT, and the image information (4) to the image information (7) is associated with the thermal invasion range in the target region LT when the treatment energy is applied to the target region LT, and the learning is performed by inputting the teacher data to the calculation model based on the multilayer neural network. Furthermore, as a method of the machine learning, for example, a method based on DNN of a multilayer neural network such as CNN or 3D-CNN is used. Furthermore, as a method of the machine learning, a method based on a recurrent neural network (RNN), an LSTM obtained by extending an RNN, or the like may be used.

Step S9 is executed in a case where it is determined that the thermal invasion range is equal to or more than the threshold (step S6: Yes).

Specifically, in step S9, the processor 48 stops the operations of the first and second power supplies 41 and 44, stops the treatment of the target region LT, and causes the notification unit 47 to notify that the thermal invasion range is equal to or more than the threshold similarly to step S7.

Step S10 is executed in a case where it is determined that the thermal invasion range is less than the threshold (step S6: No).

Specifically, in step S10, the processor 48 continuously operates the first and second power supplies 41 and 44 to continue the treatment of the target region LT.

According to the third embodiment described above, the following effects are obtained.

Since the control method (estimation method) executed by the processor 48 according to the third embodiment estimates the thermal invasion range as described above, it is possible to appropriately estimate how far the thermal invasion occurs from the jaw 7 and the treatment unit 91 in the target region LT. That is, the target region LT can be appropriately treated by the estimation.

In particular, since the thermal invasion range can be estimated while the treatment energy is applied to the target region LT, if it is predicted that the thermal invasion occurs unnecessarily, the treatment can be immediately stopped, and the target region LT can be

### Modification of Third Embodiment

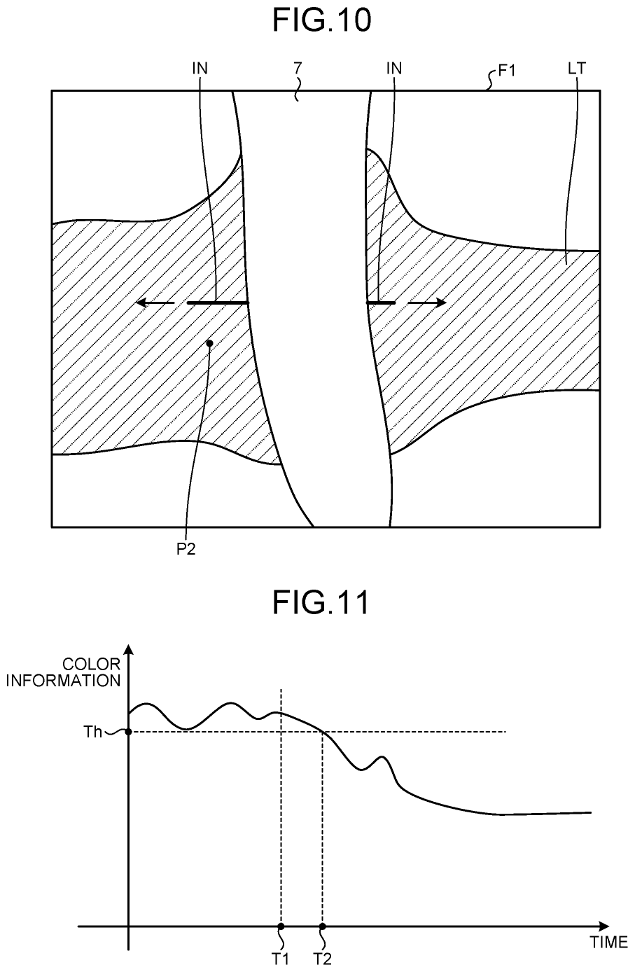

FIGS. 10 and 11 are views illustrating a modification of the third embodiment. Specifically, FIG. 10 is a view corresponding to FIG. 5. FIG. 11 is a diagram illustrating temporal changes of color information (pixel values (RGB values)) at a pixel position P2 illustrated in FIG. 5.

In the third embodiment described above, the processor 48 estimates the thermal invasion range in the target region LT by using the learning model for estimation, but the disclosure is not limited thereto, and the thermal invasion range may be estimated as in the present modification illustrated in FIGS. 10 and 11.

Specifically, the processor 48 recognizes at least one of the jaw 7 and the treatment unit 91 included as subjects in the endoscopic image F1 and a target region LT gripped between the jaw 7 and the treatment unit 91 by image recognition using the learning model for object identification stored in the storage unit 49. In addition, the processor 48 sets, as a threshold, a value obtained by subtracting the standard deviation of the color information from the average value of the color information for each pixel of the region corresponding to the target region LT on the basis of a plurality of endoscopic images captured by the imaging device 22 by the timing T1 (FIG. 11) at which the application of the treatment energy to the target region LT is started. For example, for the pixel position P2 illustrated in FIG. 10, a threshold Th (FIG. 11) is set as the threshold. Furthermore, the processor 48 constantly monitors whether or not the color information has become equal to or less than the set threshold for each pixel of the region corresponding to the target region LT on the basis of the endoscopic image F1 (FIG. 10) captured by the imaging device 22 after the application of the treatment energy to the target region LT is started. For example, the color information of the pixel position P2 illustrated in FIG. 10 becomes equal to or less than the threshold Th at a timing T2 as illustrated in FIG. 11. The timing T2 means that the thermal invasion occurs at a position corresponding to the pixel position P2 in the target region LT at the timing T2. Then, the processor 48 generates a superimposed image in which an indicator IN (FIG. 10) is superimposed on a region, in which the thermal invasion occurs, in the region corresponding to the target region LT in the endoscopic image F1, and causes the notification unit 47 (display device) to display the superimposed image. For example, in the example illustrated in FIG. 10, in a case where the thermal invasion occurs at all pixel positions arranged along the longitudinal direction (in FIG. 10, the vertical direction) of the region corresponding to at least one of the jaw 7 and the treatment unit 91 in the region corresponding to the target region LT in the endoscopic image F1, the processor 48 superimposes the indicator IN on the center position in the longitudinal direction at all the pixel positions. As a result, with the lapse of time from the start of the application of the treatment energy to the target region LT, the indicator IN superimposed on the endoscopic image F1 extends in a bar graph shape in a direction away from the region corresponding to at least one of the jaw 7 and the treatment unit 91.

As described above, in the present modification, the processor 48 estimates the thermal invasion range on the basis of the image information (color information) calculated on the basis of the endoscopic image F1.

Even in a case where the thermal invasion range is estimated as in the present modification described above, the same effects as those of the third embodiment described above are obtained.

### Fourth Embodiment

Next, a fourth embodiment will be described.

In the following description, the same reference numerals are given to the same configurations as those of the third embodiment described above, and a detailed description thereof will be omitted or simplified.

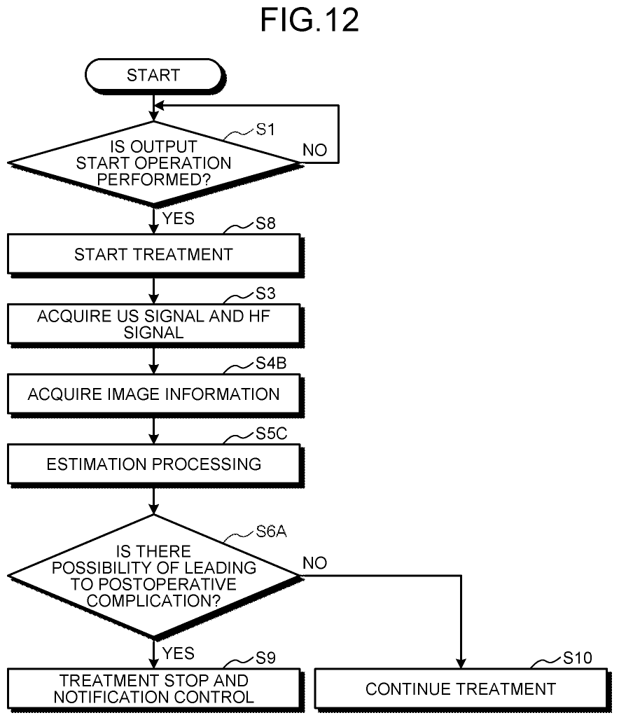

FIG. 12 is a flowchart illustrating a control method according to the fourth embodiment.

The fourth embodiment is different from the third embodiment in the estimation processing executed by the processor 48. That is, in the control method according to the fourth embodiment, as illustrated in FIG. 12, steps S5C and S6A are adopted instead of steps S5B and S6 in the control method described in the above-described third embodiment. Hereinafter, only steps S5C and S6A will be mainly described.

Step S5C is executed after step S4B.

Specifically, in step S5C, by using the learning model for estimation stored in the storage unit 49, the processor 48 uses, as input data, at least one of fifteen pieces of information of the output information (1) to the output information (11) and the image information (4) to the image information (7) and output (estimates), as output data, the possibility of leading to a postoperative complication.

Here, the learning model for estimation according to the fourth embodiment is different from the learning model for estimation described in the third embodiment described above.

The learning model for estimation according to the fourth embodiment is a learning model generated by machine learning using teacher data in which at least one of fifteen pieces of information of the output information (1) to the output information (11), which are acquired when the treatment energy is applied to the target region LT, and the image information (4) to the image information (7) is associated with the possibility of leading to a postoperative complication in a case where the treatment energy is applied to the target region LT.

Here, the learning model for estimation according to the fourth embodiment includes a neural network in which each layer includes one or a plurality of nodes. In addition, the type of machine learning is not particularly limited, but for example, it is sufficient if teacher data is prepared in which at least one of fifteen pieces of information of the output information (1) to the output information (11), which are acquired when the treatment energy is applied to the target region LT, and the image information (4) to the image information (7) is associated with the possibility of leading to a postoperative complication in a case where the treatment energy is applied to the target region LT, and the learning is performed by inputting the teacher data to the calculation model based on the multilayer neural network. Furthermore, as a method of the machine learning, for example, a method based on DNN of a multilayer neural network such as CNN or 3D-CNN is used. Furthermore, as a method of the machine learning, a method based on a recurrent neural network (RNN), an LSTM obtained by extending an RNN, or the like may be used.

Then, in a case where in step S5C, the processor 48 estimates that there is the possibility of leading to a postoperative complication (step S6A: Yes), the process proceeds to step S9.

On the other hand, in a case where in step S5C, the processor 48 estimates that there is no possibility of leading to a postoperative complication (step S6A: No), the process proceeds to step S10.

According to the fourth embodiment described above, the following effects are obtained.

Since the control method (estimation method) executed by the processor 48 according to the third embodiment estimates the thermal invasion range as described above, it is possible to appropriately estimate how far the thermal invasion occurs from the jaw 7 and the treatment unit 91 in the target region LT. That is, the target region LT can be appropriately treated by the estimation.

In particular, since the thermal invasion range can be estimated while the treatment energy is applied to the target region LT, if it is predicted that the thermal invasion occurs unnecessarily, the treatment can be immediately stopped, and the target region LT can be

### Other Embodiments

Although the embodiments for carrying out the disclosure have been described so far, the disclosure should not be limited only by the first to fourth embodiments described above.

In the first to fourth embodiments described above, the ultrasonic energy and the high frequency energy are adopted as the treatment energy applied to the target region LT, but the disclosure is not limited thereto, and only one of the ultrasonic energy and the high frequency energy may be adopted. In addition, thermal energy may be employed as the treatment energy. Note that “applying thermal energy to the target region” means transferring heat generated in a heater to the target region.

The configuration described in the first embodiment described above and the configuration described in the third embodiment described above may be combined. That is, the thermal invasion range in the target region LT may be estimated from at least one of thirteen pieces of information of the monitor information (1) to the monitor information (10) and the image information (1) to the image information (3) and at least one of fifteen pieces of information of the output information (1) to the output information (11) and the image information (4) to the image information (7).

Similarly, the configuration described in the second embodiment described above and the configuration described in the fourth embodiment described above may be combined. That is, the possibility of leading to a postoperative complication may be estimated from at least one of thirteen pieces of information of the monitor information (1) to the monitor information (10) and the image information (1) to the image information (3) and at least one of fifteen pieces of information of the output information (1) to the output information (11) and the image information (4) to the image information (7).

In the first to fourth embodiments described above, the learning model for estimation is used for the estimation of the thermal invasion range in the target region LT and the estimation of the possibility of leading to a postoperative complication, but the disclosure is not limited thereto.

For example, the processor 48 may perform the estimation of the thermal invasion range in the target region LT and the estimation of the possibility of leading to a postoperative complication by using at least one of the tissue type specified by tissue type specifying processing described below, the tissue change amount specified by tissue change amount specifying processing described below, or the heat amount specified by heat amount specifying processing described below.

The tissue type specifying processing is processing of specifying the tissue type of the target region LT.

For example, the processor 48 specifies the tissue type of the target region LT by comparing at least one of the monitor information (4), the monitor information (5), the monitor information (9), the monitor information (10), the image information (2), or the image information (3) with a specific threshold.

The tissue change amount specifying processing is processing of specifying the tissue change amount of the target region LT after the treatment energy is applied.

For example, the processor 48 specifies the tissue change amount from at least one of nine pieces of information of the output information (1), the output information (5), the output information (6), the output information (10), the output information (11), and the image information (4) to the image information (7).

The heat amount specifying processing is processing of specifying an amount of heat input to the target region LT after the treatment energy is applied.

For example, the processor 48 specifies the heat amount from at least one of nine pieces of information of the output time (1) to the output time (5), the output time (7) to the output time (9), and the image information (1).

In the first to fourth embodiments described above, in a case where the endoscopic image F1 is a fluorescence image, a region, in which fluorescence intensity (pixel value or luminance value) is equal to or more than a specific threshold, among all pixels of the fluorescence image may be estimated as a region where the thermal invasion occurs.

According to the estimation method and the estimation device according to the disclosure, it is possible to appropriately perform estimation regarding the thermal invasion in the living tissue.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

In this document the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the term “and/or” is used to refer to a nonexclusive or, such that “A and/or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. An estimation method executed by a processor of an estimation device, the estimation method comprising:
acquiring monitor information regarding an electrical characteristic value in an energy treatment tool when non-treatment energy for monitoring is applied to a living tissue from the energy treatment tool;
acquiring image information regarding an endoscopic image obtained by imaging the living tissue; and
performing an estimation regarding thermal invasion in the living tissue based on at least one of: the monitor information or the image information and on an output setting value of treatment energy for treating the living tissue with the energy treatment tool.

2. The estimation method according to claim 1, wherein the energy treatment tool applies, as the treatment energy, ultrasonic energy to the living tissue according to a supplied first drive signal, and wherein the electrical characteristic value includes an electrical characteristic value corresponding to a changed first drive signal that includes a drive signal applied after the supplied first drive signal is changed by a frequency response of the energy treatment tool.

3. The estimation method according to claim 1, wherein the energy treatment tool applies, as the treatment energy, high frequency energy to the living tissue according to a supplied second drive signal, and wherein the electrical characteristic value includes an electrical characteristic value corresponding to a changed second drive signal that includes a drive signal applied after the supplied second drive signal is changed by a frequency response of the energy treatment tool.

4. The estimation method according to claim 1, wherein the endoscopic image includes at least one of a white light image obtained by imaging the living tissue irradiated with white light, a special light image obtained by imaging the living tissue irradiated with special light in a specific wavelength band, or a fluorescence image obtained by imaging fluorescence generated from the living tissue by irradiation of the living tissue with excitation light.

5. The estimation method according to claim 1, wherein the image information includes information calculated based on the endoscopic image and is information regarding a length of a part, which is gripped by the energy treatment tool, of the living tissue.

6. The estimation method according to claim 1, wherein the image information includes color information of a pixel corresponding to the living tissue in the endoscopic image.

7. The estimation method according to claim 1, wherein the image information is information calculated based on the endoscopic image and includes information regarding at least one of a tissue structure of the living tissue, a tissue contraction amount of the living tissue after application of the treatment energy is started, or a time from start of application of the treatment energy to start of contraction of the living tissue.

8. The estimation method according to claim 1, wherein in the estimation regarding the thermal invasion in the living tissue, a thermal invasion range in the living tissue is estimated.

9. The estimation method according to claim 1, wherein in the estimation regarding the thermal invasion in the living tissue, a possibility of leading to a postoperative complication is estimated.

10. The estimation method according to claim 1, wherein in the estimation regarding the thermal invasion in the living tissue, the estimation regarding the thermal invasion in the living tissue is performed by using a learning model generated by machine learning.

11. The estimation method according to claim 1, wherein in the estimation regarding the thermal invasion in the living tissue, the estimation regarding the thermal invasion in the living tissue is performed by using at least one of a tissue type of the living tissue, a tissue change amount of the living tissue after the treatment energy is applied, or an amount of heat input to the living tissue after the treatment energy is applied.

12. The estimation method according to claim 1, wherein after an output start operation of starting application of the treatment energy to the living tissue is received, the non-treatment energy for monitoring is applied from the energy treatment tool to the living tissue and the monitor information is acquired, and wherein after the estimation regarding the thermal invasion in the living tissue is performed, when a result of the estimation is a specific result, application of the treatment energy from the energy treatment tool to the living tissue is started.

13. An estimation method executed by a processor of an estimation device, the estimation method comprising:
acquiring output information regarding an electrical characteristic value in an energy treatment tool when treatment energy is applied to a living tissue from the energy treatment tool;
acquiring image information regarding an endoscopic image obtained by imaging the living tissue; and
performing an estimation regarding thermal invasion in the living tissue based on the output information and the image information.

14. The estimation method according to claim 13, wherein after an output start operation of starting application of the treatment energy to the living tissue is received, the treatment energy is applied from the energy treatment tool to the living tissue and the output information is acquired, and wherein after the estimation regarding the thermal invasion in the living tissue is performed, when a result of the estimation is a specific result, application of the treatment energy from the energy treatment tool to the living tissue is continued.

15. An estimation device comprising:
at least one processor, the at least one processor being configured to:
acquire monitor information regarding an electrical characteristic value in an energy treatment tool when non-treatment energy for monitoring is applied to a living tissue from the energy treatment tool;
acquire image information regarding an endoscopic image obtained by imaging the living tissue; and
perform an estimation regarding thermal invasion in the living tissue based on at least one of the monitor information and the image information and on an output setting value of treatment energy for treating the living

16. The estimation device according to claim 15, wherein the energy treatment tool applies, as the treatment energy, ultrasonic energy to the living tissue according to a supplied first drive signal, and wherein the electrical characteristic value includes an electrical characteristic value corresponding to a changed first drive signal that includes a drive signal applied after the supplied first drive signal is changed by a frequency response of the energy treatment tool.

17. The estimation device according to claim 15, wherein the energy treatment tool applies, as the treatment energy, high frequency energy to the living tissue according to a supplied second drive signal, and wherein the electrical characteristic value includes an electrical characteristic value corresponding to a changed second drive signal that includes a drive signal applied after the supplied second drive signal is changed by a frequency response of the energy treatment tool. tissue with the energy treatment tool.

18. An estimation device comprising:
at least one processor, the at least one processor being configured to:
acquire output information regarding an electrical characteristic value in an energy treatment tool when treatment energy is applied to a living tissue from the energy treatment tool;
acquire image information regarding an endoscopic image obtained by imaging the living tissue; and
perform an estimation regarding thermal invasion in the living tissue based on the output information and the image information.

19. The estimation device according to claim 18, wherein after an output start operation of starting application of the treatment energy to the living tissue is received, the treatment energy is applied from the energy treatment tool to the living tissue and the output information is acquired, and wherein after the estimation regarding the thermal invasion in the living tissue is performed, when a result of the estimation is a specific result, application of the treatment energy from the energy treatment tool to the living tissue is continued.
